(19) United States
(12) Patent Application Publication
(10) Pub. No.: US 2009/0234384 A1
(43) Pub. Date: Sep. 17, 2009

(54) Absorbable surgical materials

(76) Inventor: Ahmad R. Hadba, Wallingford, CT (US)

Correspondence Address: Tyco Healthcare Group LP, 60 MIDDLETOWN AVENUE, NORTH HAVEN, CT, 06473, US

(21) Appl. No.: 11/990,794

(22) PCT Filed: Aug. 25, 2006
(86) PCT No.: PCT/US06/33452

§ 371 Date: Feb. 20, 2008

Related U.S. Application Data
(60) Provisional application No. 60/711,730, filed on Aug. 26, 2005.

(51) Int. Cl. A61B 17/04; A61B 17/03; A61B 17/06

(52) U.S. Cl. 606/215; 606/230; 606/224

(57) ABSTRACT

The present disclosure provides absorbable surgical materials.

### BACKGROUND

1. Technical Field

The present disclosure relates to absorbable surgical materials and more particularly to absorbable metallic surgical materials, such as filaments.

2. Background of Related Art

Absorbable materials are known in the art for a variety of uses, particularly those uses in which the absorbable material is implanted within a living organism for medical purposes. Such materials are useful for temporarily holding tissues in a desired position during healing, and being absorbed by the organism after a period of time.

Absorbable materials, i.e., filaments, are usually made from non-metallic materials such as natural or synthetic polymers or copolymers and resins, including protein based-materials. However, non-metallic materials often are not stiff enough to penetrate certain tissue without the assistance from metallic pins, drills and the like.

Filaments that may include inert metallic materials, such as stainless steel, are not typically absorbable. Therefore they are frequently permanently implanted or used on wound sites that allow the metallic filament to be removed from the wound site after the wound has sufficiently healed.

Thus, the need exists for absorbable metallic surgical filaments that maintain sufficient stiffness to penetrate tissue, maintain sufficient tensile strength during the healing process and produce no harmful effects to the body when absorbed.

### SUMMARY

The present disclosure provides a surgical filament and a method of using the filament. The surgical filament comprises a combination of metal materials that dissolve in a human body without any harmful effects. The method of using the filament includes approximating the tissue surrounding a wound and affixing a surgical filament described herein to the approximated tissue.

### BRIEF DESCRIPTION OF THE DRAWING

FIG. 1 illustrates a surgical needle suture combination according to the present disclosure.

### DETAILED DESCRIPTION

The present disclosure provides a surgical filament comprising a combination of metal materials which dissolve in the human body without any harmful effects on the person who wears the implant. The combination of metal materials is to be designed such that the material of the surgical filament dissolves at a certain decomposition rate and without the production of bio-incompatible decomposition products. A surgical filament of this type combines the advantageous mechanical properties of metallic surgical filaments with the bioabsorbability of non-metallic, or polymer-based surgical filaments.

In one embodiment, the combination of metal materials is a metal alloy, the selection of the alloy constituents—as explained in detail below—serving to attain the prerequisite of biocompatible dissolution of the alloy. Consequently, the metal alloy has to consist of a combination of material that will dissolve in the body comparatively rapidly—within a period of some months—forming harmless constituents.

For correspondingly uniform dissolution to be obtained, such an alloy comprises a first component which covers itself with a protective oxide coat. This first component is selected from one or several metals of the group of magnesium, titanium, zirconium, niobium, tantalum, zinc or silicon. For uniform dissolution of the mentioned oxide coat to be attained, a second component is added to the alloy, possessing sufficient solubility in blood or interstitial fluid, such as lithium sodium, potassium, calcium, iron or manganese.

The mentioned elements are suitable because they are present in the human body anyway—such as magnesium, zinc, sodium, potassium, calcium, iron and manganese—or are known to be nontoxic—such as titanium, zirconium, niobium, tantalum, silicon and lithium. The combination of a passivating and a soluble component ensures a timely and uniform dissolution into biocompatible breakdown products. The dissolution rate can be regulated through the ratio of the two components.

In a particularly useful embodiment, the alloy is to be formed so that the dissolution products are soluble salts, such as sodium, potassium, calcium, iron or zinc salts, or that non-soluble dissolution products, such as titanium, tantalum or niobium oxide originate as colloidal particles. The dissolution rate is adjusted by way of the composition so that gases, such as hydrogen which evolves during the dissolution of lithium, sodium, potassium, magnesium, calcium or zinc, dissolve physically, not forming any macroscopic gas bubbles.

One combination of alloys that is particularly useful is a sodium-magnesium alloy. Since sodium hydroxide as a dissolution product possesses a high solubility, this alloy dissolves without voluminous encrusting. Sodium dissolves and magnesium hydroxide forms a fine precipitate which may deposit without risk in the wound healing tissue.

Another useful combination of metal materials is a zinc-titanium alloy, the percentage by weight of which is in the range of 0.1% to 1%. This combination precludes the comparatively strong crystalline growth of zinc as a material used, which would cause a comparatively brittle and fragile behavior of the surgical fastener. When the material is worked, the addition of titanium leads to the formation of a Zn₁₅Ti phase on the crystal boundaries which precludes any further crystalline growth. This reduction of the grain size generally improves the ductility, in particular the elongation at rupture—i.e. the percentage elongation of the material under mechanical load as far as to the rupture thereof.

If gold is added to this alloy at a percentage by weight of 0.1% to 2%, a further reduction of the grain size is attained when the material cures. This further improves the tensile strength of the material.

In addition to the realization of the combination of metal materials in the form of a metal alloy, another fundamental embodiment may be the design of the combination of metal materials as an electrochemical local element. On the one hand, this element consists of a substantially pure metal which constitutes the body of the surgical filament and on the other hand, of a local electrode of a second metal which is in contact therewith. The local electrode combines with the filament to form a local element in which the potential of the filament is displaced, corresponding to the electron-chemical series. The contact voltage originating causes the corrosion process of the filament. The corrosion rate and thus the decomposition rate of the filament can be controlled by way of the size of the surface of contact between the corrodible filament and the local electrode connected thereto or by the selection of the participant element.

In keeping with two alternative embodiments, the participant element can be a coat on the support body or an individual metal part attached to the support body for example by welding. In this case, the local electrodes may also serve as X-ray markers, having a typical double function.

The absorbable metallic filaments may be fabricated by a number of known processing techniques. Some examples include, spinning, extruding, casting, injection molding and blow molding. In short any processing technique capable of creating filaments is contemplated to form the present absorbable filaments.

The surgical filaments disclosed herein may be used to form various surgical devices. Some examples include monofilament sutures, multifilament sutures, sternum closure devices, meshes, slings, tissue scaffolds, anti-adhesion barriers, vascular grafts, and bone fillers. The surgical devices may be formed from at least one filament that can be braided, woven or non-woven. In a particularly useful embodiment at least one filament is braided to form surgical devices such as multifilament sutures, sternum closure devices, meshes, slings and anti-adhesion barriers.

In yet another embodiment the braided surgical device may consist of two or more filaments. The two or more filaments may comprise the same or different metals or alloys. The use of two or more different metallic filaments can be used to generate an electrochemical cell.

A surgical braid is a configuration containing at least two surgical yarns mechanically blended by intertwining the yarns in a braided construction. The yarns are continuous and discrete, so therefore each yarn extends substantially along the entire length of the braid and maintains its individual integrity during braid preparation, processing and use. See, commonly owed, co-pending U.S. application Ser. No. 10/972,464, the disclosure of which is herein incorporated by reference.

Similarly, a surgical yarn is a configuration containing at least two surgical filaments mechanically bundled together to form a yarn. The filaments are continuous and discrete, so therefore each filament extends substantially along the entire length of the yarn and maintains its individual integrity during yarn preparation, processing and use.

A plurality of similar surgical filaments is used to form a homogenous surgical yarn and a plurality of dissimilar surgical filaments is used to form a heterogeneous surgical yarn. A plurality of similar surgical yarns is used to form a homogeneous surgical braid and a plurality of dissimilar surgical yarns is used to form a heterogeneous surgical braid.

In one embodiment, a heterogeneous surgical yarn contains a plurality of two dissimilar surgical filaments. A first surgical filament is made from an absorbable metallic material as described herein and a surgical second filament is made from any absorbable or non-absorbable material other than the absorbable metal material in the first surgical filament. A plurality of the two dissimilar surgical filaments are commingled to form a heterogeneous surgical yarn.

In another embodiment, a heterogeneous surgical braid contains two dissimilar surgical yarns. A first surgical yarn contains a plurality of surgical filaments made from an absorbable metallic material as described herein. A second surgical yarn contains a plurality of surgical filaments made from any absorbable or non-absorbable material other than the absorbable metal material in the first surgical filament. The first and second surgical yarns are in direct intertwining contact to form a heterogeneous surgical braid.

In still another embodiment, a heterogeneous surgical braid contains a heterogeneous surgical yarn and a homogeneous surgical yarn. As described above, a heterogeneous surgical yarn contains a plurality of two dissimilar surgical filaments. A first surgical filament is made from an absorbable metal material as described herein and a second surgical filament is made from any absorbable or non-absorbable material other than the absorbable metal material in the first surgical filament. A homogeneous surgical yarn contains a plurality of surgical filaments made from any material capable of being spun into a filament. The heterogeneous surgical yarn and the homogeneous surgical yarn are in direct intertwining contact to form a heterogeneous surgical braid.

In yet another embodiment, a surgical braid contains two similar heterogeneous surgical yarns. Each heterogeneous surgical yarn contains a plurality of two dissimilar surgical filaments. A first surgical filament is made from an absorbable metal material as described herein and a second surgical filament is made from any absorbable or non-absorbable material other than the absorbable metal material in the first surgical filament. Each heterogeneous surgical yarn is in direct intertwining contact to form a surgical braid.

Additionally, it is envisioned that the yarns and braids described herein may also contain two or more dissimilar filaments containing two or more dissimilar metals or alloys that are commingled or intertwined to form an electrochemical cell. The dissimilar metals or alloys may be selected based upon a desired degradation rate. Also, the degradation of the electrochemical cell may create a localized leak current, which can be used to promote wound healing via electrical or electrochemical stimulation.

Some examples of absorbable and non-absorbable materials include, but are not limited to, natural, synthetic, biodegradable, non-biodegradable and shape memory polymeric materials. A particularly useful polymeric material may be selected from the group consisting of polyamides, polyesters, polyacrylonitrile, polyethylene, polypropylene, polyglycolic acid, polylactic acid, polydioxanone, polyepsilon-caprolactone, and polytrimethylene carbonate.

Representative natural biodegradable polymers include polysaccharides such as alginate, dextran, cellulose, collagen, and chemical derivatives thereof (substitutions, additions of chemical groups, for example, alkyl, alkylene, hydroxylations, oxidations, and other modifications routinely made by those skilled in the art), and proteins such as albumin, zein and copolymers and blends thereof, alone or in combination with synthetic polymers.

Representative synthetic polymer blocks include polyphosphazenes, poly(vinyl alcohols), polyamides, polyester amides, poly(amino acid)s, synthetic poly(amino acids), polyanhydrides, polycarbonates, polyacrylates, polyalkylenes, polyacrylamides, polyalkylene glycols, polyalkylene oxides, polyalkylene terephthalates, polyortho esters, polyvinyl ethers, polyvinyl esters, polyvinyl halides, polyvinylpyrrolidone, polyesters, polylactides, polyglycolides, polysiloxanes, polyurethanes and copolymers thereof.

Examples of suitable polyacrylates include poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate) and poly(octadecyl acrylate).

Synthetically modified natural polymers include cellulose derivatives such as alkyl celluloses, hydroxyalkyl celluloses, cellulose ethers, cellulose esters, nitrocelluloses, and chitosan. Examples of suitable cellulose derivatives include methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate and cellulose sulfate sodium salt. These are collectively referred to herein as “celluloses”.

Representative synthetic degradable polymers include polyhydroxy acids, such as polylactides, polyglycolides and copolymers thereof; poly(ethylene terephthalate); poly(hydroxybutyric acid); poly(hydroxyvaleric acid); poly[lactide-co-(ε-caprolactone)]; poly[glycolide-co-(ε-caprolactone)]; polycarbonates, poly(pseudo amino acids); poly(amino acids); poly(hydroxyalkanoate)s; polyanhydrides; polyortho esters; and blends and copolymers thereof.

Examples of non-biodegradable polymers include ethylene vinyl acetate, poly(meth)acrylic acid, polyamides, polyethylene, polypropylene, polystyrene, polyvinyl chloride, polyvinylphenol, and copolymers and mixtures thereof.

Rapidly bioerodible polymers such as poly(lactide-co-glycolide)s, polyanhydrides, and polyorthoesters, which have carboxylic groups exposed on the external surface as the smooth surface of the polymer erodes, also can be used.

A non-absorbable shape memory alloy is also capable of being spun into a continuous filament and combined with the absorbable metallic filaments. Some examples of shape memory alloys include, but are not limited too, nitinol (TiNi), CuZnAl, CuAlNi and FeNiAl. A particularly useful alloy is nitinol (TiNi). A non-absorbable shape memory alloy possesses the ability to remember its original shape, either after mechanical deformation, which is a one-way effect, or by cooling and heating, which is a two-way effect. This phenomenon is based on a structural phase transformation which is known as martensitic transformation.

A surgical braid and/or yarn can be prepared using conventional braiding technology and equipment commonly used in the textile industry, and in the medical industry for preparing multifilament sutures. Suitable braid constructions are disclosed, for example, in U.S. Pat. Nos. 3,187,752; 3,565,077; 4,014,973; 4,043,344; 4,047,533; 5,019,093; and 5,059,213, the disclosures of which are incorporated herein by reference. Illustrative flat braided structures (suitable, e.g., for tendon repair) which can be formed using the presently described surgical yarns include those described in U.S. Pat. Nos. 4,792,336 and 5,318,575. Suitable mesh structures are shown and described, for example, in Hain et al. U.S. Pat. No. 5,292,328. In addition, absorbable metallic filaments may be incorporated into non-woven structures, such as felt. One suitable non-woven structure is shown and described in Koyfman et al. U.S. Pat. No. 5,393,534.

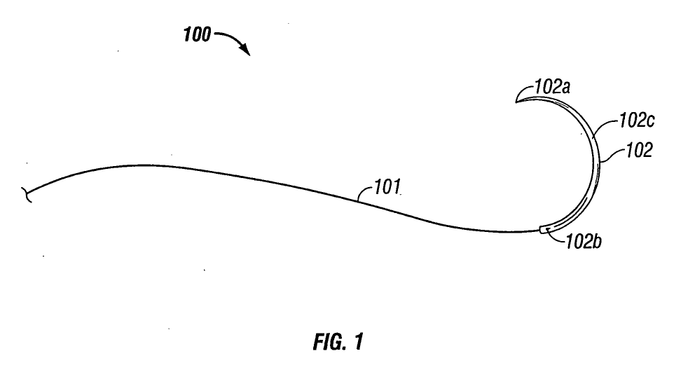

As stated above, the homogeneous or heterogeneous surgical braids and yarns may be used to form a variety of surgical devices. It is envisioned that the surgical braids and yarns described herein are used to form multifilament sutures, as shown in FIG. 1.

A suture in accordance with the present disclosure may be attached to a surgical needle 100 as shown in FIG. 1 by methods well known in the art. Wounds may be sutured by approximating tissue and passing the needle suture through or around the tissue to create wound closure. If present, the needle is then removed from the suture and the suture is secured (e.g., knotted, tied off, etc.)

In another embodiment, the absorbable surgical material described herein may be used to form at least part of the needle 102. The configuration of the present absorbable suture is not critical. A wide variety of surgical needles are known in the art, including, but not limited to needles having bodies that are square, rectangular, oval star-shaped or the like.

The needle may be sharp or blunt or may have one or more cutting edges. Needles in accordance with the present disclosure can be made using convection techniques such as stamping, coining, grinding, polishing, bending and the like.

As those skilled in the art will appreciate, surgical needle typically consists of three basic parts: the first end 102a which is the tip that is used to penetrate tissue, the body 102c of the needle that is shaped according to the intended use of the needle and the second end 102b which is used to receive and/or attach the suture 101. During the closing of a wound or the implanting of a surgical device, needles have been reported to break, chip, shatter, etc. for a variety of different reasons, such as, for example, unintended or unexpected contact with bone. A needle made from the absorbable metallic material as described herein may remain in the tissue and dissolve over time inflicting no harm to the patient.

In yet another embodiment, the absorbable filaments may be used to form sternum closure devices. Sternum closure devices are known and are used routinely to following surgeries involving a median sternotomy, e.g., open heart surgery, to secure the approximated sternum halves in place. Some examples of sternum closure devices include braided sutures, and metal wire cables.

It is further envisioned that the surgical filaments described herein and the surgical devices made from these filaments would be used in conjunction with other surgically biocompatible wound treatment materials that include, adhesives whose function is to attach or hold organs, tissues or structures; sealants to prevent fluid leakage; hemostats to halt or prevent bleeding; and medicaments. Examples of adhesives which can be employed include protein derived, aldehyde-based adhesive materials, for example, the commercially available albumin/glutaraldehyde materials sold under the trade designation BioGlue™ by Cryolife, Inc., and cyanoacrylate-based materials sold under the trade designations Indermil™ and Derma Bond™ by Tyco Healthcare Group, LP and Ethicon Endosurgery, Inc., respectively. Examples of sealants, which can be employed, include fibrin sealants and collagen-based and synthetic polymer-based tissue sealants. Examples of commercially available sealants are synthetic polyethylene glycol-based, hydrogel materials sold under the trade designation CoSeal™ by Cohesion Technologies and Baxter International, Inc. Examples of hemostat materials, which can be employed, include fibrin-based, collagen-based, oxidized regenerated cellulose-based and gelatin-based topical hemostats. Examples of commercially available hemostat materials are fibrinogen-thrombin combination materials sold under the trade designations CoStasis™ sold by Orthovita, Inc. and Tisseel™ sold by Baxter International, Inc. Hemostats herein include astringents, e.g., aluminum sulfate, and coagulants.

The adhesive, sealant or medicament may be disposed on or impregnated into any of the surgical fasteners described herein. The medicament may include one or more medically and/or surgically useful substances such as drugs, enzymes, growth factors, peptides, proteins, dyes, diagnostic agents or hemostasis agents or any other pharmaceutical used in the prevention of stenosis.

It is also contemplated that sutures in accordance with this disclosure may be formed in conjunction with a suture anchor delivery system and may be passed through tissue using an arthroscopic suturing instrument. Such suture anchor delivery systems and arthroscopic suturing instruments are within the purview of one skilled in the art.

Various modifications and variations of the embodiments described herein will be apparent to those skilled in the art from the foregoing detailed description. Such modifications and variations are intended to come within the scope of the following claims.

What is claimed is:

1. A medical device comprising:
at least one filament comprising a combination of metal materials that dissolve in a human body.

2. The medical device of claim 1 wherein the combination of metal materials is a metal alloy comprising of a first component which forms a protecting passiviation coat and a second component which ensures sufficient corrosion of the alloy.

3. The medical device of claim 2 wherein the first component is at least one metal selected from the group consisting of magnesium, titanium, zirconium, niobium, tantalum, zinc and silicon.

4. The medical device of claim 2 wherein the second component is at least one metal selected from the group consisting of lithium, sodium, potassium, manganese, calcium and iron.

5. The medical device of claim 2 wherein the metal alloy contains magnesium.

6. The medical device of claim 2 wherein the metal alloy is a magnesium-sodium alloy.

7. The medical device of claim 1 further comprising an additional component selected from the group consisting of tissue adhesive, tissue sealant and medicament.

8. The medical device of claim 1 wherein the medical device is a suture.

9. The medical device of claim 1 wherein the medical device is a multifilament suture.

10. The multifilament suture of claim 9 further comprising at least one polymeric filament.

11. The multifilament suture of claim 10 wherein the at least one polymeric filament is bioabsorbable.

12. A surgical yarn comprising at least a first and second surgical filament in a braided construction wherein at least one of the first and second surgical filaments comprises a surgical filament of claim 1; and
optionally, the second surgical filament comprises an absorbable or non-absorbable material that is dissimilar to the surgical filament of claim 1.

13. A surgical braid comprising at least a first and second surgical yarn in a braided construction wherein at least one of the first and second surgical yarn comprises the surgical yarn of claim 12; and
optionally, the second surgical yarn comprises an absorbable or non-absorbable surgical filament that is dissimilar to the yarn of claim 12.

14. A surgical yarn comprising at least a first and second surgical filament in a braided construction wherein the first surgical filament comprises a surgical filament of claim 1; and
the second surgical filament comprises a dissimilar metal surgical filament of claim 1.

15. The surgical yarn of claim 14 wherein the two dissimilar metal filaments are chosen to generate a current during corrosion to stimulate the wound healing process.

16. A method of closing a wound comprising the steps of:
approximating two tissue surfaces; and
securing the tissue surfaces using a medical device according to claim 1.

17. The method of closing a wound in claim 16 further comprising the step of:
applying an additional component selected from the group consisting of tissue adhesive, tissue sealant and medicament to the secured, approximated tissue.

18. A surgical needle comprising:
a tissue penetration portion; and
a suture attachment portion, wherein the surgical needle is made at least in part from a combination of metal materials that dissolves in a human body.

19. The surgical needle of claim 18 wherein the combination of metal materials is a metal alloy comprising of a first component which forms a protecting passivation coat and a second component which ensures sufficient corrosion of the alloy.

20. The surgical needle of claim 18 wherein the first component is at least one metal selected from the group consisting of magnesium, titanium, zirconium, niobium, tantalum, zinc and silicon.

21. The surgical needle of claim 18 wherein the second component is at least one metal selected from the group consisting of lithium, sodium, potassium, manganese, calcium and iron.

22. The surgical needle of claim 18 wherein the metal alloy contains magnesium.

23. The surgical needle of claim 18 wherein the metal alloy is a magnesium-sodium alloy.
